(19) United States
(12) Patent Application Publication
(10) Pub. No.: US 2009/0038973 A1
(43) Pub. Date: Feb. 12, 2009

(54) Package with anatomically correct product placement

(75) Inventor: Daniel D. Feldman, Brooklyn, NY (US)

Correspondence Address: Novak, Druce & Quigg LLP, 1300 I Street, N.W., Suite 1000, West Tower, WASHINGTON, DC, 20005, US

(73) Assignee: Profoot, Inc., Brooklyn, NY (US)

(21) Appl. No.: 11/882,886

(22) Filed: Aug. 6, 2007

(51) Int. Cl. A61B 19/00

(52) U.S. Cl. 206/438

(57) ABSTRACT

A package for packaging medical/cosmetic products. The package is made of opaque material which is sealed to prevent contact with the packaged product. One or more cutouts are made in these opaque packaging to permit contact with the packaged product without opening the package. An optional transparent film may separate the cutout opening from the product.

### BACKGROUND OF THE INVENTION

1. Field of the Invention

The invention relates to packages, more specifically packages containing medical or cosmetic products, such as bandages, moleskin, corn removers, adhesive pads, gauze and other similar products designed to be applied to a portion of the human anatomy. In a particularly preferred embodiment, the packages are designed to contain products to be applied to the human torso or extremities, such as a human arm or in such a case a foot, and the package is provided with a printed indicia or replication of the human anatomy, such as a foot. A window in the package exposes the product within the package. By locating the window, which can take the form of a cutout in the package in relation to the indicia of the human anatomy depicted on the package, the consumer of the product will understand how to apply the product in the intended area and in the correct orientation to the intended area of the anatomy. If the specific area of the anatomy to be covered need to be cut from a larger supply of the product in the package, the size, shape and location of the window/cutout in the package will assist the consumer in obtaining the size/shape of a specific portion of the supply of material.

2. Description of the Related Art

While it is true that current medical terminology can precisely identify the location of medical/cosmetic products to be applied to the human anatomy, many of the current population cannot understand such medical terms. Such lack of understanding may be caused by several factors such as lack of education, English as a second language, or lack of English skills at all. Further, although illustrations in packaging inserts could be provided, the potential consumer would not be able to read the insert until the product was purchased, which may result in the purchase of products inappropriate for the desired use. Furthermore, any packaging inserts would increase the cost of the product and any written information in the packaging material would have to explain to the consumer how to cut and/or shape the product from a supply of the product in the package, in order to obtain the appropriately shaped and sized product for specific anatomical application. In addition, the instructions would then need to inform the consumer as to the correct orientation of the product to the portion of human anatomy to which it is to be applied.

Thus, the present invention avoids these problems of the prior art.

### BRIEF SUMMARY OF THE INVENTION

The present invention provides a closed and sealed package for medical or cosmetic materials for topical application to the human anatomy. In a particularly preferred embodiment, the package including medical/cosmetic materials are for use on the human foot and may include bandages, moleskin, corn removers, corn cushions, callous cushions and similar materials for self-application by a consumer.

The package preferably contains an indicia of the human anatomy to which the medical/cosmetic product is to be applied. The package is generally opaque thereby concealing the contents of the package except for windows, which may take the form of cutouts, in the package exposing the contents of the package in relation to the anatomical indicia on the package.

These window/cutout depicts not only the location to which the product is to be self-applied by the consumer, but also provides an indication of the shape and/or size or the product to be applied, such that it can be cut from a larger supply of product in the package, as well as the orientation of the product to the anatomy, e.g., the so-called butterfly bandages/pads for use at elbows, knees, knuckles, etc.

The cutout permits the consumer to actually feel the product without opening the package, and in the case of products such as cushions or moleskin, provides the consumer with a sense of the quality of the product in the package.

Although, the package has been described with reference to the human foot, it is equally applicable for packaging other medical/cosmetic products for topical application to all parts of the human anatomy.

These and other objects and advantages of the present invention will become evident from the appended drawings and the detailed description of the invention.

### BRIEF DESCRIPTION OF THE DRAWINGS

FIG. 1 illustrates a package intended for containing a callous cushion having a cutout portion positioned on a printed indicia of the bottom of the human foot;

FIG. 2 illustrates an alternative embodiment of the invention in which a package intended to contain moleskin is provided with several windows/cutouts to expose the product, which windows/cutouts are positioned in proximity to the upper surface of a human foot;

FIG. 3 is a still further embodiment of the invention in which a package intended to contain products for use with corns on a human foot is provided with windows/cutouts so as to view and access directly the product within the package;

FIG. 4 is a sectional view along lines 4-4 of FIG. 1;

FIG. 5 is a sectional view alone lines 5-5 of FIG. 2; and

FIG. 6 is a sectional view along lines 6-6 of FIG. 3.

### DETAILED DESCRIPTION OF THE PREFERRED EMBODIMENTS

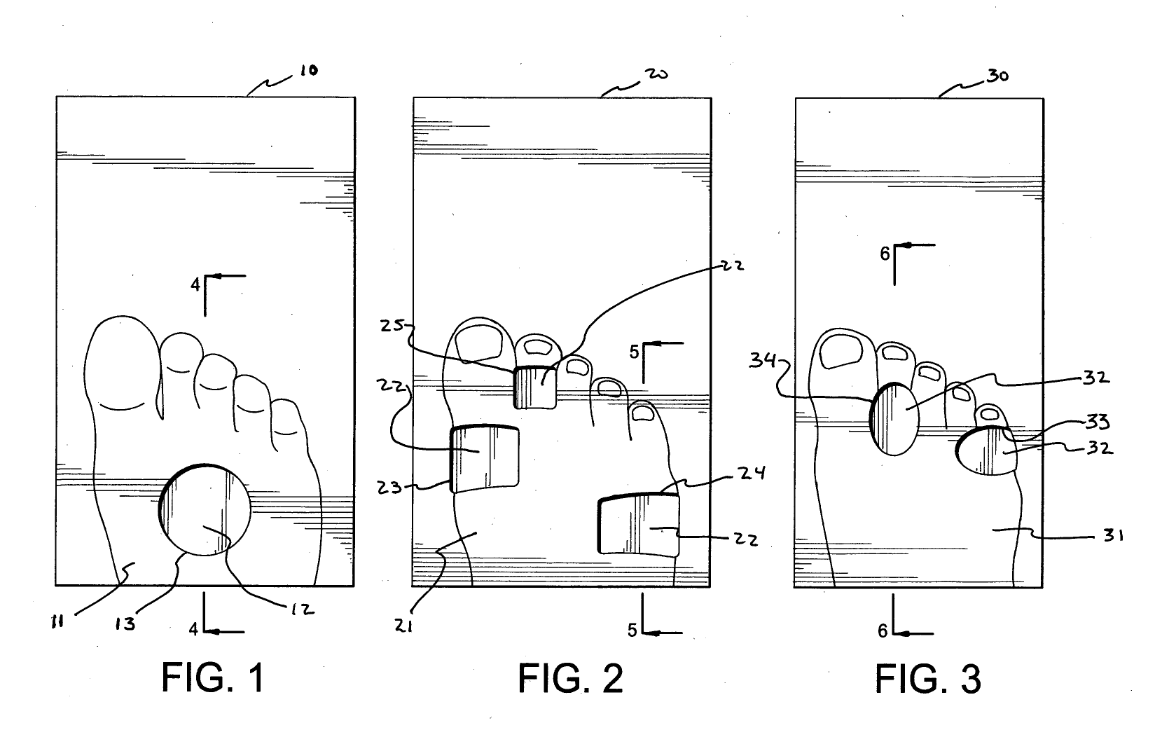

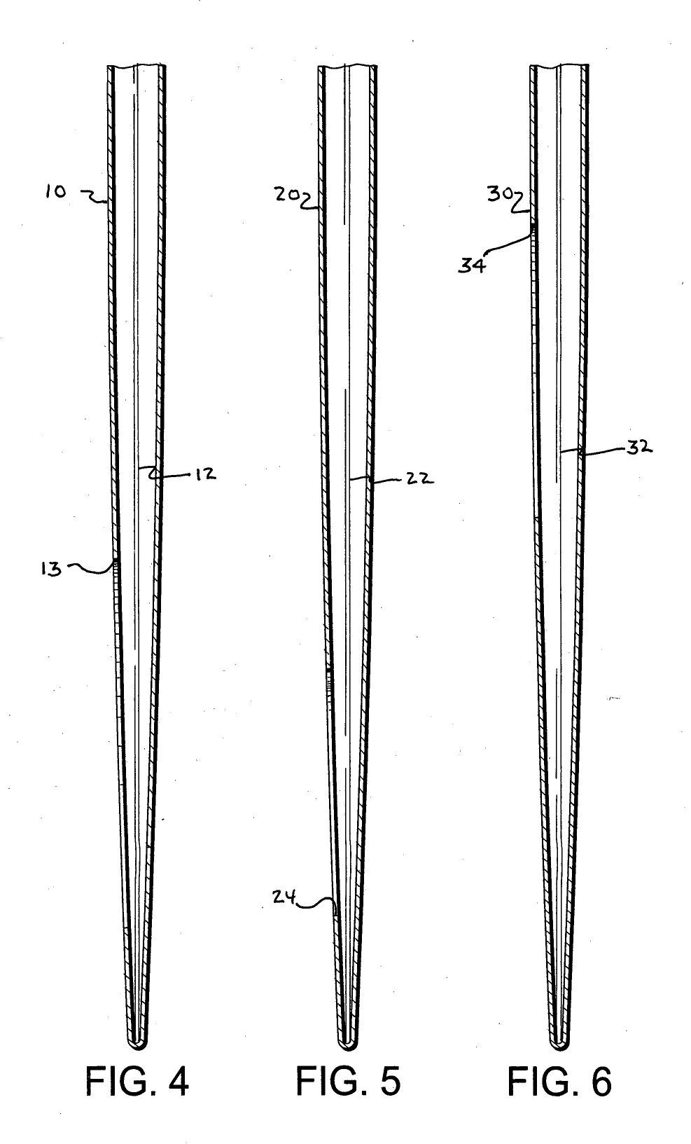

As shown in FIG. 1, package 10, made of an opaque material, such as paper, cardboard or opaque plastic is printed with indicia 11, in this case depicting the bottom of a human foot. Inside the package 10 is a medical/cosmetic material such as a callous cushion 12 viewable and accessible to touch by virtue of a cutout 13 in package 1. The cutout 13 can be formed with a knife or scored during manufacture of package 10 to create a opening, exposing the cushion 12. Not only does the shape of cutout 13 provide the consumer with information as to the shape of the portion to be cut from a supply of cushion 12, but the cutout which is placed in relation to indicia 11 of the bottom of a foot informs the consumer as to where and how to self-apply the product to his own foot. The cutout 13 also permits the consumer to directly touch the cushion 12 thereby judging its qualities, without having to open package 10. If desired for regulatory reasons a transparent window (not shown) or wrapper (not shown) may be placed between the product 12 and the consumer, at least in the areas of cutout 13, to prevent consumer contact with the product 12.

In an alternative embodiment of the invention depicted in FIG. 2 a package 20 containing a product 22, such as moleskin, may be visible through one or more cutouts 23, 24, 25 superimposed upon indicia 21 depicting the upper surface of a human foot. These cutouts 23, 24, 25 may each vary from each other not only in location on indicia 21 2, but also in size and shape. As with the embodiment of FIG. 1 the cutouts 23, 24, 25 permits the consumer to directly touch the product 22 to assess its qualities without opening the package 20. As with the previous embodiment of FIG. 1, where regulations require, the cutouts 23, 23, 25 may each be providing with a transparent window (not shown) or one window (not shown) may extend across two or more of cutouts 23, 24, 25 or around the product 22 to prevent the consumer from physically contacting product 22.

In a still further embodiment of the invention as shown in FIG. 3, a package 30 containing a product 32, such as corn cushions, or corn removers, and containing indicia 31 depicting an upper surface of a human foot may be provided with one or more cutouts 33, 34 to permit a consumer to assess the qualities of the product 32 by direct contact therewith through cutout 33 and/or 34 without opening package 30. Again, as with the embodiments of FIGS. 1 and 2 where regulation, local custom or other requirements prohibit the consumer from directly touching product 32, a transparent window (not shown) may be placed over one or both cutouts 33, 34 or the product 32 wrapped in a transparent sleeve (not shown) or covered with a transparent film to permit viewing of the product 32 while preventing direct contact therewith by the consumer.

Having fully described my invention, it will be readily apparent that he invention is readily adaptable to a wide range of uses but depicting other parts of the anatomy, e.g. bandages for knees or elbows; wart removal pads for hands, etc. without depicting from the spirit and scope of the appended claims.

I claim:

1. A package, said package being formed of an opaque material;
said package being sealed to enclose a medical or cosmetic product;
said package being provided with at least one indicia depicting at least a portion of a human anatomy
said package having a cutout therein to make the product contained within said package visible;
said cutout being placed in relation to said indicia to simulate the placement of said product on the human anatomy.

2. The package of claim 1, further containing a transparent film between the cutout and the product.

3. The package of claim 1 having at least two cutouts.

4. The package of claim 3 further comprising a transparent film between each of the cutouts and the product.

5. The package of claim 2 wherein the transparent film envelops the product.

6. The package of claim 1 wherein the product is moleskin.

7. The package of claim 1 wherein the cutout permits consumer contact with the product without opening the sealed package.

8. The package of claim 1 further comprising a product which is a callous cushion.

9. The package of claim 1 further comprising a product which is moleskin.

10. The package of claim 1 further comprising a product which is a corn remover.

11. The package of claim 1 further comprising of a product which is a corn pad.

12. The package of claim 1 wherein the portion of human anatomy depicted is a human foot.

13. A packaged product, said product being packaged in a sealed package made of opaque material and containing an indicia of at least a portion of human anatomy, said sealed package having at least one cutout therein to permit viewing of the product in relationship to the indicia of human anatomy.

14. The packaged product of claim 13 wherein said opaque material is formed of paper or cardboard.

15. The package product of claim 13 wherein the product is at least one selected from the group consisting of bandage, callus cushions, moleskin, corn cushions and corn removers.

16. The packaged product of claim 13 wherein the cutout permits contact by the consumer with the packaged product without opening the sealed package.

17. The packaged product of claim 13 further comprising a transparent film between the product and cutout.

18. The packaged product of claim 13 wherein the indicia is that of a human foot.

19. The packaged product of claim 13 having at least two cutouts.

20. The packaged product of claim 19 wherein each of the cutouts differs in at least one property of shape or size from the other cutout.
